(19) United States
(12) Patent Application Publication
(10) Pub. No.: US 2007/0054921 A1
(43) Pub. Date: Mar. 8, 2007

(54) Substituted indoles and a process for preparing substituted indoles

(76) Inventor: Ian W Davies, Princeton, NJ (US)

Correspondence Address: MERCK AND CO., INC, P O BOX 2000, RAHWAY, NJ, 07065-0907, US

(21) Appl. No.: 10/557,537

(22) PCT Filed: Jun. 1, 2004
(86) PCT No.: PCT/US04/17357

§ 371 Date: Nov. 21, 2005

Related U.S. Application Data
(60) Provisional application No. 60/476,089, filed on Jun. 5, 2003.

(51) Int. Cl. A61K 31/496; A61K 31/4709; A61K 31/4439; C07D 403/14; C07D 403/02

(52) U.S. Cl. 514/253.07; 514/314; 514/312; 514/414; 514/337; 544/363; 546/153; 546/277.4

(57) ABSTRACT

The instant invention is directed to novel compounds of Formulae (I) and (II), as wells a process for preparing compounds of Formula (II). The process comprises a palladium-catalyzed reductive cyclization of a compound of Formula (I) to produce a compound of Formula (II).

### BACKGROUND OF THE INVENTION

Synthesis of 2-substituted indoles generally relies upon cross-coupling reactions of an appropriately functionalized and protected indole. In 1997, Söderberg et al reported the palladium-catalyzed reductive cyclization of 2-nitrostyrenes using 6 mol % Pd(OAc)₂ and 24 mol % PPh₃ under 60 psi CO at 70° C. (Söderberg, B. C., Shriver, J. A., J. Org. Chem., 1997, 62, 5838-5845; Söderberg, B. C., Rector, S. R, O'Neil, S. N., Tetrahedron Lett., 1999, 40, 3657-3660; Söderberg, B. C., Chisnell, A. C., O'Neil, S. N., Shriver, J. A., J. Org. Chem., 1999, 64, 9731-9734; Scott, T. L., Söderberg, B. C., Tetrahedron Lett. 2002, 43, 1621-1624) The use of 5 mol % PdCl₂(PPh3)₂ to effect this transformation was reported by Watanabe et al, however, the addition of 50 mol % SnCl₂ is required for reasonable reaction rate. (Akazome, M.; Kondo, T., Watanabe, Y., J. Org. Chem., 1994, 59, 3375-3380). The high catalyst, ligand, and additive loading prohibit large scale application of this chemistry. The catalyst system employed by Cenini et al required high pressure (300-900 psi), temperature (120° C.) and catalyst loading (5 mol %) and led to substantial dimer formation (up to 10%). (Tollari, S., Cenini, S., Crotti, C., Gianella, E. J. Molecular Catalysis 1994, 87, 203-214; Ragaini, F., Sportiello, P., Cenini, S. J. Orgmet. Chem. 1999, 577, 283-291.)

The process of the instant invention, which utilizes a palladium-catalyzed reductive cyclization of ortho-nitrostyrenes, possesses several advantages. This process can be conducted using milder conditions, such as lower temperatures and CO pressure, as well as lower catalyst and ligand loading, which simplifies purification of the indole product.

### SUMMARY OF THE INVENTION

The instant invention is directed to novel compounds of Formula I and Formula II and to a process for preparing substituted indoles of Formula II. The process comprises a palladium-catalyzed reductive cyclization of a compound of Formula I to produce a compound of Formula II.

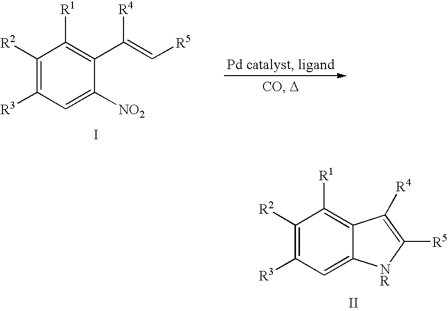

### DETAILED DESCRIPTION OF THE INVENTION

The present invention relates to novel compounds that are synthetic intermediates of pharmaceutical compounds, such as KDR inhibitors or GNRH inhibitors. In a first embodiment, the instant invention is directed to compounds illustrated by Formula I:

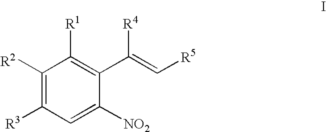

wherein

R^{a} is independently selected from a) hydrogen, and b) unsubstituted or substituted C₁-C₆ alkyl;

R¹ is a) hydrogen, b) unsubstituted or substituted C₁-C₆ alkyl, and c) OR⁷;

R² is a) hydrogen, b) unsubstituted or substituted C₁-C₆ alkyl, c) (CR^{a}₂)ₙR⁷, d) O(CR^{a}₂)ₙOR⁷, e) O(CR^{a}₂)ₙR⁷, or f) halo;

R³ is a) hydrogen, b) unsubstituted or substituted C₁-C₆ alkyl, or c) OR⁷;

R² and R³ can be taken together to form a cyclic moiety, (CH₂)ᵤ, said cyclic moiety optionally containing one or two heteroatoms selected from N, O and S;

R⁴ is a) hydrogen, b) unsubstituted or substituted C₁-C₆ alkyl, c) OR⁷, or d) C(O)₂R⁷;

R⁵ is a) unsubstituted or substituted C₁-C₆ alkyl, b) C₂-C₆ alkenyl-R⁷, c) C₂-C₆ alkynyl-R⁷, d) unsubstituted or substituted aryl, e) unsubstituted or substituted heterocyclyl, f) C(O)NR⁷(CR^{a}₂)ₙC(O)OR⁷, or g) C(O)R⁷; said alkyl, alkenyl, alkynyl, aryl or heterocyclyl is optionally substituted with at least one substituent selected from: i) halo, ii) unsubstituted or substituted C₁-C₆ alkyl, iii) OR⁷, iv) NR⁷₂, v) NO₂, and vi) S(O)ₘR⁶;

R⁶ is independently selected from a) unsubstituted or substituted C₁-C₆ alkyl, and b) unsubstituted or substituted aryl;

R⁷ is independently selected from a) H, b) unsubstituted or substituted C₁-C₆ alkyl, c) unsubstituted or substituted aryl, d) unsubstituted or substituted heterocyclyl, and e) CF₃; said alkyl, aryl and heterocyclyl is optionally substituted with at least one substituent selected from i) halo, ii) unsubstituted or substituted C₁-C₆ alkyl, iii) OR⁷, iv) NR⁷₂, v) NO₂, and vi) S(O)ₘR⁶,

m is 1 or 2;

n is independently 0, 1, 2, 3, or 4;

u is 4, 5, 6, 7 or 8;

or a salt thereof.

In a further embodiment of the first embodiment, the instant invention is a compound of Formula I, as described above, or a salt thereof, wherein:

R¹ is hydrogen;

R⁴ is a) hydrogen, or b) C(O)₂R⁷;

and all other substituents and variable are as defined above.

Specific examples of compounds of Formula I include:
Trans-3-{2-[5-(4-methanesulfonyl-piperazine-1-ylmethyl)-2-nitro-phenyl]-vinyl}-2-methoxy-quinoline;
Methyl-N-[(2E)-3-6-nitro-1,3-benzodioxol-5-yl)prop-2-enoyl]glycinate;
(2E)-3-2-nitrophenyl)-1-phenylprop-2-en-1-one;
(2E)-3-(2-nitrophenyl)acrylaldehyde;
2-Nitro-1-[(1E)-prop-1-en-1-yl]-4-(trifluoromethoxy)benzene;
2-Methoxy-5-[(E)-2-(5-methoxy-2-nitrophenyl)vinyl]pyridine;
2-Methoxy-3-[(E)-2-(5-methyl-2-nitrophenyl)vinyl]pyridine;
2-Chloro-3-[(E)-)-2-[5-(2-methoxyethoxy)-2-nitrophenyl]vinyl}quinoline;
2-Methoxy-3-{(E)-2-[5-(2-methoxyethoxy)-2-nitrophenyl]vinyl}quinoline;
2-Methoxy-3-[(E)-2-[2-nitro-5-(2-piperidin-1-ylethoxy)phenyl]vinyl}quinoline;
2-Chloro-3-[(E)-2-(5-methyl-2-nitrophenyl)vinyl]quinoline;
2-Methoxy-3-[(E)-2-5-methyl-2-nitrophenyl)vinyl]quinoline;
3-[(E)-2-(5-{[4-(methylsulfonyl)piperazin-1-yl]methyl}-2-nitrophenyl)vinyl]quinolin-2-(1H)-one;
2-[(E)-2-(5-chloro-2-nitrophenyl)vinyl]-1-(phenylsulfonyl)-1H-indole;
Methyl (2Z)-2-[2-nitro-4-(trifluoromethoxy)phenyl]-3-phenylacrylate;
1,1′-(1E,3E)-buta-1,3-diene-1,4-diylbis(2-nitrobenzene);
   or a salt thereof.

In a second embodiment, the instant invention is directed to compounds illustrated by Formula II:

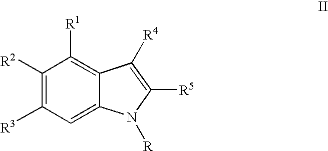

wherein

R is H or OH;

R^{a} is independently selected from a) hydrogen, and b) unsubstituted or substituted C₁-C₆ alkyl;

R¹ is a) hydrogen, b) unsubstituted or substituted C₁-C₆ alkyl, and c) OR⁷;

R² is a) hydrogen, b) unsubstituted or substituted C₁-C₆ alkyl, c) (CR^{a}₂)ₙR⁷, d) O(CR^{a}₂)ₙOR⁷, e) O(CR^{a}₂)ₙR⁷, or f) halo;

R³ is a) hydrogen, b) unsubstituted or substituted C₁-C₆ alkyl, or c) OR⁷;

R² and R³ can be taken together to form a cyclic moiety, (CH₂)ᵤ, said cyclic moiety optionally containing one or two heteroatoms selected from N, O and S;

R⁴ is a) hydrogen, b) unsubstituted or substituted C₁-C₆ alkyl, c) OR⁷, or d) C(O)₂R⁷;

R⁵ is a) unsubstituted or substituted C₁-C₆ alkyl, b) C₂-C₆ alkenyl-R⁷, c) C₂-C₆ alkynyl-R⁷, d) unsubstituted or substituted aryl, e) unsubstituted or substituted heterocyclyl, or f) C(O)NR⁷(CR^{a}₂)ₙC(O)OR⁷; said alkyl, alkenyl, alkynyl, aryl or heterocyclyl is optionally substituted with at least one substituent selected from: i) halo,

ii) unsubstituted or substituted C₁-C₆ alkyl, iii) OR⁷, iv) NR⁷₂, v) NO₂, and vi) S(O)ₘR⁶;

R⁶ is independently selected from a) unsubstituted or substituted C₁-C₆ alkyl, and b) unsubstituted or substituted aryl;

R⁷ is independently selected from a) H, b) unsubstituted or substituted C₁-C₆ alkyl, c) unsubstituted or substituted aryl, d) unsubstituted or substituted heterocyclyl, and e) CF₃; said alkyl, aryl and heterocyclyl is optionally substituted with at least one substituent selected from i) halo, ii) unsubstituted or substituted C₁-C₆ alkyl, iii) OR₇, iv) NR⁷₂, v) NO₂, and vi) S(O)ₘR⁶,

m is 1 or 2;

n is independently 0, 1, 2, 3, or 4;

u is 4, 5, 6, 7 or 8;

or a pharmaceutically acceptable salt thereof.

In a further embodiment of the second embodiment, the instant invention is a compound of Formula II, as described above, or a pharmaceutically acceptable salt thereof, wherein

R¹ is hydrogen;

R⁴ is hydrogen or C(O)₂R⁷;

R⁵ is a) unsubstituted or substituted C₁-C₆ alkyl, b) unsubstituted or substituted aryl, c) unsubstituted or substituted heterocyclyl, or d) C(O)NR⁷CR^{a}₂)ₙC(O)OR⁷;

and all other substituents and variables are as defined above.

Examples of compounds of Formula II include:
2-Methoxy-3-[5-(piperazin-1-ylmethyl)-1H-indol-2-yl]quinoline;
N-(Carbomethoxy)-5,6-methylenedioxy-1H-indole-2-carboxamide;
2-(2-methoxyquinolin-3-yl)-6-methyl-5-{[4-(methylsulfonyl)piperazin-1-yl]methyl}-1H-indol-1-ol;
2-Methoxy-6-[5-methoxy-1H-indol-2-yl] pyridine;
2-Methoxy-3-[5-methyl-1H-indol-2-yl] pyridine;
2-Chloro-3-[5-(methoxyethoxy)-1H-indol-2-yl]quinoline;
2-Methoxy-3-[5-(methoxyethoxy)-1H-indol-2-yl]quinoline;
2-Methoxy-3-[5-(1-piperdinylethoxy)-1H-indol-2-yl]quinoline;
2-Chloro-3-5-methyl-1H-indol-2-yl)quinoline;
2-Methoxy-3-(5-methyl-1H-indol-2-yl)quinoline;
3-[5-[4-(Methylsulfonyl)-1-piperazinyl]methyl]-1H-indole-2-yl]quinolin-2(1H)-one;
1-Benzenesulfonyl-2-(1′benzyl-5-chloroindol-2′-yl) indole;
Methyl 2-phenylindole-3-carboxylate;
   or a pharmaceutically acceptable salt thereof.

Specific examples of compounds of Formula II include:
2-(2-methoxyquinolin-3-yl)-6-methyl-5-{[4-(methylsulfonyl)piperazin-1-yl]methyl}-1H-indol-1-ol; and
2-Methoxy-3-[5-[[4-(methysulfonyl)-1-piperazinyl]methyl]-1H-indol-2-yl]-quinoline
   or a pharmaceutically acceptable salt thereof.

A third emodiment of the instant invention is directed to the synthesis of the compound of the Formula II, as described above, which comprises a palladium-catalyzed reductive cyclization of an ortho-nitrostyrene of Formula I:

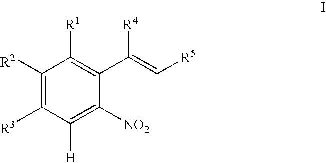

wherein R¹, R², R³, R⁴ and R⁵ are as defined above, to produce a compound of Formula II.

In a further embodiment of the third embodiment of the instant invention, the palladium catalyst utilized is generated in situ. The palladium catalyst can be formed in situ utilizing a palladium source and a ligand.

In another embodiment of the third embodiment, the palladium catalyst utilized is preformed.

A specific embodiment of the instant invention is a process for preparing 2-(2-methoxyquinolin-3-yl)-6-methyl-5-{[4-(methylsulfonyl)piperazin-1-yl]methyl}-1H-indol-1-ol which comprises
a) mixing trans-3-{2-[5-(4-methanesulfonyl-piperazine-1-ylmethyl)-2-nitro-phenyl]-vinyl}-2-methoxy-quinoline with a palladium catalyst and a solvent to produce a reaction mixture;
b) pressurizing the reaction mixture to about 15 psig with CO and maintaining a temperature of about 70° C.; and
c) isolating 2-(2-methoxyquinolin-3-yl)-6-methyl-5-{[4-(methylsulfonyl)piperazin-1-yl]methyl}-1H-indol-1-ol.

Another specific embodiment of the instant invention is a process for preparing 2-methoxy-3-[5-[[4-(methysulfonyl)-1-piperazinyl]methyl]-1H-indol-2-yl]-quinoline which comprises
a) mixing trans-3-{2-[5-(4-methanesulfonyl-piperazine-1-ylmethyl)-2-nitro-phenyl]-vinyl}-2-methoxy-quinoline with a palladium catalyst, a aromatic diamine and a solvent to produce a reaction mixture;
b) pressurizing the reaction mixture to about 15 psig with CO and maintaining a temperature of about 70° C.; and
c) isolating 2-methoxy-3-[5-[[(4-methysulfonyl)-1-piperazinyl]methyl]-1H-indol-2-yl]-quinoline.

The compounds of the present invention may have asymmetric centers, chiral axes, and chiral planes (as described in: E. L. Eliel and S. H. Wilen, Stereochemistry of Carbon Compounds, John Wiley & Sons, New York 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable or substituent (e.g. R¹, n, etc.) occurs more than one time in any constituent, its definition on each occurrence is independent at every other occurrence. Also, combinations of substituents and variables are permissible only if such combinations result in stable compounds.

Lines drawn into the ring systems from substituents indicate that the indicated bond may be attached to any of the substitutable ring carbon atoms or heteroatoms, including the carbon atom or heteroatom that is the point of attachment. If the ring system is polycyclic it is intended that the bond may be attached to any of the suitable carbon atoms or heteroatoms of any ring.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

As used herein, “alkyl” is intended to include both branched and straight-chain aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, propyl, isopropyl, butyl, t-butyl, pentyl, hexyl, heptyl, octyl, nonyl, decyl, and so on.

“Cycloalkyl” as used herein is intended to include non-aromatic cyclic hydrocarbon groups, having the specified number of carbon atoms, which may or may not be bridged or structurally constrained. Examples of such cycloalkyls include, but are not limited to, cyclopropyl, cyclobutyl, cyclopentyl, cyclohexyl, adamantyl, cyclooctyl, cyclopentyl, tetrahydro-naphthalene, methylenecylohexyl, and the like. As used herein, examples of “C₃-C₁₀ cycloalkyl” may include, but are not limited to:

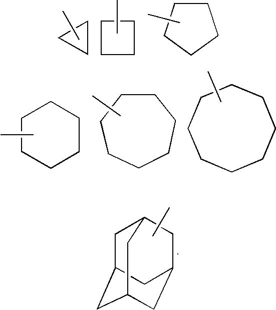

As used herein, the term “alkoxy” represents an alkyl group of indicated number of carbon atoms attached through an oxygen bridge.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to ally, the straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl, indanonyl, indenyl, biphenyl, tetralinyl, tetralonyl, fluorenonyl, phenanthryl, anthryl, acenaphthyl, tetrahydronaphthyl, and the like. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl; carbazoyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro, fluoro, bromo and iodo.

The term heterocycle or heterocyclic or heterocyclyl, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. “Heterocycle” or “heterocyclyl” therefore includes the above mentioned heteroaryls, as well as dihydro and tetrathydro analogs and N-oxides thereof. Further examples of “heterocyclyl” include, but are not limited to the following: benzodioxolyl, benzofuranyl, benzofurazanyl, benzimidazolyl, benzopyranyl, benzopyrazolyl, benzotriazolyl, benzothiazolyl, benzothienyl, benzothiofuranyl, benzothiophenyl, benzothiopyranyl, benzoxazolyl, carbazolyl, carbolinyl, chromanyl, cinnolinyl, diazapinonyl, dihydrobenzodioxinyl, dihydrobenzofuranyl, dihydrobenzofuryl, dihydrobenzoimidazolyl, dihydrobenzothienyl, dihydrobenzothiopyranyl, dihydrobenzothiopyranyl sulfone, dihydrobenzothiophenyl, dihydrobenzoxazolyl, dihydrocyclopentapyridinyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxanyl, dioxolanyl, dioxidotetrahydrothienyl, dioxidothiomorpholinyl, furyl, furanyl, imidazolyl, imidazolinyl, imidazolidinyl, imidazothiazolyl, imidazopyridinyl, indazolyl, indolazinyl, indolinyl, indolyl, isobenzofuranyl, isochromanyl, isoindolyl, isoindolinyl, isoquinolinone, isoquinolyl, isothiazolyl, isothiazolidinyl, isoxazolinyl, isoxazolyl, methylenedioxybenzoyl, morpholinyl, naphthpyridinyl, oxadiazolyl, oxazolyl, oxazolinyl, oxetanyl, oxidothiomorpholinyl, oxoazepinyl, oxadiazolyl, oxodihydrophthalazinyl, oxodihydroindolyl, oxodihydrotriazolyl, oxoimidazolidinyl, oxopiperazinyl, oxopiperdinyl, oxopyrrolidinyl, oxopyrimidinyl, oxopyrrolyl, oxotriazolyl, piperidyl, piperidinyl, piperazinyl, pyranyl, pyrazinyl, pyrazolyl, pyridazinyl, pyridinonyl, pyridopyridinyl, pyridazinyl, pyridyl, pyridinyl, pyrimidinyl, pyrrolyl, pyrrolidinyl, quinazolinyl, quinolinyl, quinolyl, quinolinonyl, quinolinone, quinoxalinyl, tetrahydrobenzoannulenyl, tetrahydrocycloheptapyridinyl, tetrahydrofuranyl, tetrahydrofuryl, tetrahydroisoquinolinyl, tetrahydropyranyl, tetrahydroquinolinyl, tetrazolyl, tetrazolopyridyl, thiadiazolyl, thiazolyl, thiazolinyl, thienofuryl, thienyl, triazolyl, azetidinyl, 1,4-dioxanyl, hexahydroazepinyl, and the like. Attachment of a heterocyclyl substituent can occur via a carbon atom or via a heteroatom.

In an embodiment of the instant invention, heterocyclyl is selected from oxoazepinyl, benzimidazolyl, dioxanyl, dioxolanyl, dioxanyl, dioxidotetrahydrothienyl, oxetanyl, piperidinyl, pyrazolyl, pyridinyl, tetrahydrofuranyl, tetrahydropyranyl, imidazolyl, morpholinyl, piperidyl, piperazinyl, pyridyl, pyrrolidinyl, oxopiperidinyl, oxopyrrolidinyl, quinolinyl, tetrahydrofuryl, and N-oxides thereof. In a further embodiment of the instant invention, heterocyclyl is selected from pyridinyl, quinolinyl, quinolinone, or indolyl. In a further embodiment, heterocyclyl is pyridinyl, quinolinyl or quinolinone.

As used herein, “aralkyl” is intended to mean an aryl moiety, as defined above, attached through a C₁-C₁₀ alkyl linker, where alkyl is defined above. Examples of aralkyls include, but are not limited to, benzyl, naphthylmethyl and phenylpropyl.

As used herein, “heterocyclylallyl” is intended to mean a heterocyclic moiety, as defined below, attached through a C₁-C₁₀ alkyl linker, where alkyl is defined above. Examples of heterocyclylalkyls include, but are not limited to, pyridylmethyl, imidazolylethyl, pyrrolidinylmethyl, morpholinylethyl, quinolinylmethyl, imidazolylpropyl and the like.

As used herein, the terms “substituted C₁-C₁₀ alkyl” and “substituted C₁-C₆ alkoxy” are intended to include the branch or straight-chain alkyl group of the specified number of carbon atoms, wherein the carbon atoms may be substituted with 1 to 3 substituents selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆ alkyl)O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heterocycle, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

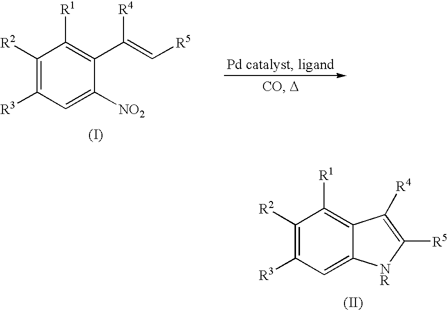

As used herein, the terms “substituted C₃-C₁₀ cycloalkyl”, “substituted aryl”, “unsubstituted phenyl”, “substituted heterocycle”, “substituted aralkyl” and “substituted heterocyclylalkyl” are intended to include the cyclic group containing from 1 to 3 substituents in addition to the point of attachment to the rest of the compound. Preferably, the substituents are selected from the group which includes, but is not limited to, halo, C₁-C₂₀ alkyl, CF₃, NH₂, N(C₁-C₆ alkyl)₂, NO₂, oxo, CN, N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl) OC(O)NH—, aryl, aralkyl, heteroaryl, heterocyclylalkyl, halo-aryl, halo-aralkyl, halo-heterocycle, halo-heterocyclylalkyl, cyano-aryl, cyano-aralkyl, cyano-heterocycle and cyano-heterocyclylalkyl.

As used herein, the phrase “substituted with at least one substituent” is intended to mean that the substituted group being referenced has from 1 to 6 substituents. Preferably, the substituted group being referenced contains from 1 to 3 substituents, in addition to the point of attachment to the rest of the compound.

In an embodiment of the instant invention, R⁵ of Formula II is selected from unsubstituted or substituted C₁-C₆ alkyl, unsubstituted or substituted phenyl, unsubstituted or substituted pyridinyl, unsubstituted or substituted quinolinyl, unsubstituted or substituted quinolinone, unsubstituted or substituted indole, or C(O)NR⁷CR^{a}₂)ₙC(O)OR⁷. In a further embodiment, R⁵ of Formula II is selected from unsubstituted or substituted C₁-C₆ alkyl, unsubstituted or substituted phenyl, unsubstituted or substituted pyridinyl, unsubstituted or substituted quinolinyl, unsubstituted or substituted quinolinone, or C(O)NR⁷CR^{a}₂)ₙC(O)OR⁷.

The salts of Formula I of the instant invention include conventional salts of Formula I compounds that may be formed using inorganic and organic acids that have a pKa less than about 4. For example, the salts include those derived from inorganic acids such as hydrochloric, hydrobromic, sulfuric, sulfamic, phosphoric, nitric, hexafluorophosphate, perchlorate, tetrafluoroborate, hexafluroroantimonate, tetraarylborates and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention include the conventional non-toxic salts of the compounds of this invention as formed inorganic or organic acids. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Abbreviations, which may be used in the description of the chemistry and in the Examples that follow, include:
Ac2O (Acetic anhydride); AcOH (Acetic acid); AIBN (2,2′-Azobisisobutyronitrile); Ar (Aryl); BINAP (2,2′-Bis(diphenylphosphino)-1,1′ binaphthyl); Bn (Benzyl); BOC/Boc (tert-Butoxycarbonyl); BSA (Bovine Serum Albumin); CAN (Ceric Ammonia Nitrate); CBz (Carbobenzyloxy); CI (Chemical Ionization); DBAD (Di-tert-butyl azodicarboxylate); DBU (1,8-Diazabicyclo[5.4.0]undec-7-ene); DCC (1,3 Dichlorohexylcarbodiimide); DCE (1,2-Dichloroethane); DCM (Dichloromethane); DIEA (N,N-Diisopropylethylamine); DMAP(4-Dimethylaminopyridine); DMA (Dimethylacetamide); DME (1,2-Dimethoxyethane); DMF (N,N-Dimethylformamide); DMSO (Methyl sulfoxide); DPPA (Diphenylphosphoryl azide); DTT (Dithiothreitol); EDC (1-(3-Dimethylaminopropyl)-3-ethyl-carbodiimide-hydrochloride); EDTA (Ethylenediaminetetraacetic acid); ELSD (Evaporative Light Scattering Detector); ES (Electrospray); ESI (Electrospray ionization); Et₂O (Diethyl ether); Et₃N (Triethylamine); EtOAc (Ethyl acetate); EtOH (Ethanol); FAB (Fast atom bombardment); HEPES (4-(2-Hydroxyethyl)-1-piperazineethanesulfonic acid); HMPA (Hexamethylphosphoramide); HOAc (Acetic acid); HOBT (1-Hydroxybenzotriazole hydrate); HOOBT (3-Hydroxy-1,2,2-benzotriazin-4(3H)-one); HPLC (High-performance liquid chromatography); HRMS (High Resolution Mass Spectroscopy); KOtBu (Potassium tert-butoxide); LAH (Lithium aluminum hydride); LCMS (Liquid Chromatography Mass Spectroscopy); MCPBA (m-Chloroperoxybenzoic acid); Me (Methyl); MeOH (Methanol); Ms (Methanesulfonyl); MS (Mass Spectroscopy); MsCl (Methanesulfonyl chloride); n-Bu (n-butyl); n-Bu₃P (Tri-n-butylphosphine); NaHMDS (Sodium bis(trimethylsilyl)amide); NBS (N-Bromosuccinimide); NMM (N-methylmorpholine); NMR (Nuclear Magnetic Resonance); Pd (Palladium); Pd(PPh₃)₄(Palladium tetrakis(triphenylphosphine)); Pd₂(dba)₃ (Tris(dibenzylideneacetone)dipalladium (O)); Ph (Phenyl); PMSF (α-Toluenesulfonyl fluoride); PS-DCC (Polystyrene dicyclohexylcarbodiimide); PS-DMAP (Polystyrene dimethylaminopyridine); PS-NMM (Polystyrene N-methylmorpholine); Py or pyr (Pyridine); PYBOP (Benzotriazol-1-yloxytripyrrolidinophosphonium) (or PyBOP) (hexafluorophosphate); RPLC (Reverse Phase Liquid Chromatography); RT (Room Temperature); SCX SPE (Strong Cation Exchange Solid Phase Extraction); t-Bu (tert-Butyl); TBAF (Tetrabutylammonium fluoride); TBSCl (tert-Butyldimethylsilyl chloride); TFA (Trifluoroacetic acid); THF (Tetrahydrofuran); TIPS (Triisopropylsilyl); TLC (Thin layer chromatography); TMS (Tetramethylsilane); and Tr (Trityl).

Substituted indoles are privileged structures that are present in a wide range of pharmacophores including KDR kinase inhibitors. (Evans, B. E. et al. J. Med. Chem. 1988, 31, 2235.) An embodiment of the instant invention is directed to a method to rapidly and efficiently access these compounds. The catalyst system comprising Pd(OAc)₂ and 1,10-phenanthroline has been used for the industrial scale reductive carbonylation of nitroaromatic compounds to isocyanates and carbamates Wehman, P.; Kaasjager, V. E.; de Lange, W. G. J.; Jartl, F.; Kamer, P. C. J.; van Leeuwen, P. W. N. M. Organometallics 1995, 14, 3751-3761; Wehman, P.; Kamer, P. C. J.; van Leeuwen, P. W. N. M. Chem. Comm. 1996, 217-218; Wehman, P.; Borst, L.; Kamer, P. C. J.; van Leeuwen, P. W. N. M. J. Mol. Cat. A: Chem. 1996, 112, 23-26; Paul, F.; Fischer, J.; Ochensbein, P; Osborn, J. A. Organometallics 1998, 17, 2199-2206; Paul, F.; Fischer, J.; Ochensbein, P; Osborn, J. A. C. R. Chimie 5 2002, 267-287.

The catalyst system of the instant invention is highly effective in the reductive cyclization of compounds of Formula I. Compounds of Formula I can be used to synthesize pharmaceutical compounds, such as 3-(5{[4-(Methylsulfonyl)piperzin-1-yl]methyl}-1H-indol-2-yl)quinolin-2(1H-one, which is claimed in U.S. Pat. No. 6,306,874 and is herein incorporated by reference. Unexpectedly, the cyclization of the instant process utilized to synthesize compounds of Formula I occurs under much milder conditions and at lower catalyst loadings than previously reported. The catalyst system of the instant invention is also highly effective in the absence of phosphine, in contrast to processes reported in the literature. Importantly, the catalyst/ligand loading and CO pressure can be decreased without adversely effecting the yield. These advantages make the reductive cyclization of ortho-nitrostyrenes a viable route to indoles on a large scale.

The reaction of the instant invention can be performed under a range of conditions. “Palladium-catalyzed” means that a palladium catalyst is used in the instant invention. Both in situ generated palladium catalysts and preformed palladium catalysts effect the transformation. An in situ generated palladium catalyst utilizes a palladium source, in conjunction with an appropriate ligand, to form the catalyst system. Sources of palladium include palladium (O) complexes, include, but are not limited to, Pd₂(dba)₃, and palladium (II) salts. Types of palladium (II) salts include, but are not limited to palladium (II) acetate, palladium (II) trifluoroacetate, and palladium (II) triflate. Appropriate ligands that can be utilized include, but are not limited to, aromatic diamines, semicorrins, and bisoxazolines. Types of aromatic diamines include, but are not limited to, 1,10-phenanthroline (phen), 3,4,7,8-tetramethyl-1,10-phenanthroline, and bipyridine. With the in situ palladium catalyst system, palladium loading can vary from about 0.05 to about 1.5 mol % with ligand loading in the range of about 0.2 to about 25 mol %.

In another embodiment of the instant invention, preformed palladium catalysts, including phen₂Pd(OTf)₂, phen₂Pd(PF₆)₂ and phen₂Pd(BF₄)₂, may be utilized to promote cyclization. The reaction occurs in the presence of additives such as Ag(OTf)₂, and Cu(OAc)₂. Suitable solvents include dimethylformamide, DMSO, THF, acetonitrile, toluene, dimethylacetamide, N-methyl pyrrolidinone, and ortho-dichlorobenzene. When a preformed palladium catalyst is utilized, the pressure for the reaction can vary from about 5 to about 90 psig CO. In a specific embodiment of the instant invention, the pressure is about 5 to about 40 psig CO. In a further embodiment, the pressure is about 15 to about 20 psig CO. For process of the instant invention, the temperatures can range from about 30° C. to about 110° C. In a specific embodiment, the temperature is between about 40° C. to about 70° C. In a specific embodiment of the instant invention, the conditions for the cyclization of the instant invention, regardless of whether the catalyst is generated in situ or is preformed, occur at a pressure of about 15 psi CO and a temperature about 70° C. The concentration of the ortho-nitrostyrene of Formula I can range from about 5 to about 300 g/L. In a specific embodiment, the concentration range is from about 30 to about 150 g/L.

A variety of functionality is tolerated by these new reductive cyclization conditions. Compounds of Formula I, including α,β-Unsaturated amides, ketones, aldehydes, methoxy- and chloro-substituted quinolines, pyridines, (Z)-alkenes and (E)/(Z)-mixtures, can be used in the instant process to form compounds of Formula II. The chemistry of the instant invention can also be applied for the synthesis of other compounds, such as those described in patents and patent publications, including US Patent 2002/0041880A1 and U.S. Pat. No. 6,306,874 B1, which are herein incorporated by reference.

### Schemes

The process of the instant invention can be utilized to prepare KDR inhibitors (such as those described in U.S. Pat. No. 6,306,874) and GNRH compounds. The following schemes, in addition to other standard manipulations that are known in the literature or exemplified in the experimental procedures, illustrate the process for preparing compounds of Formula I and Formula II. These schemes, therefore, are not limited by the compounds listed or by any particular substituents employed for illustrative purposes.

One approach to the requisite ortho-nitrostyrenes involves addition of trimethylsilylmethyl-substituted nitroaromatic compounds to aldehydes followed by elimination (Scheme 1).

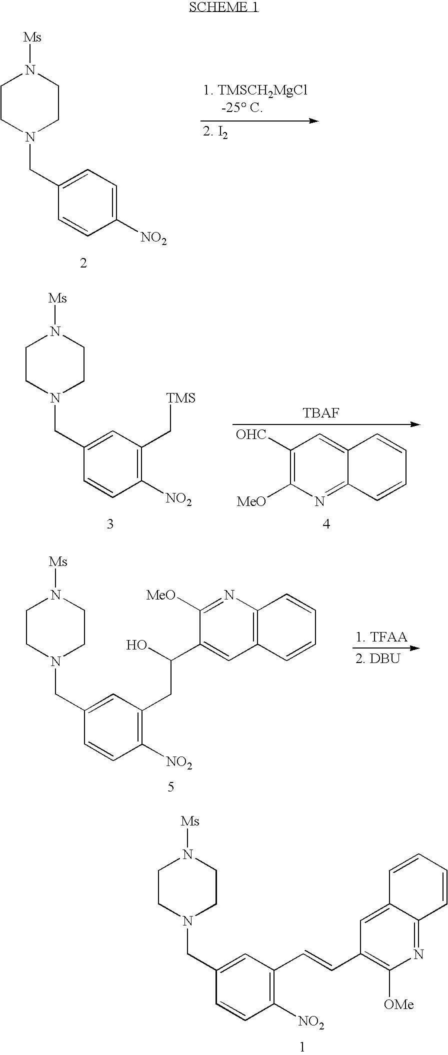

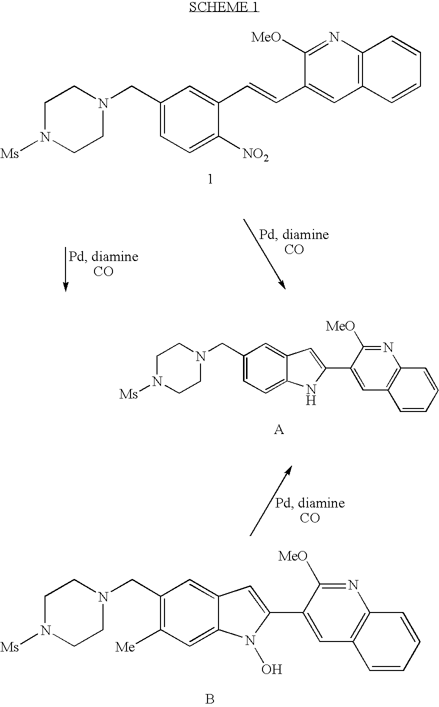

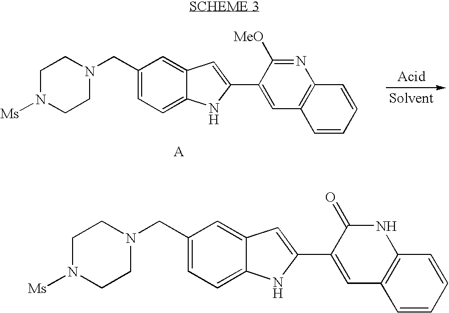

### EXAMPLES

Examples provided are intended to assist in a further understanding of the invention. Particular materials employed, species and conditions are intended to be illustrative of the invention and not limiting of the reasonable scope thereof.

Melting points are uncorrected. All solvents and reagents were used as received from commercial sources. Analytical samples were obtained by chromatography on silica gel using an ethyl acetate-hexane mixture as the eluent unless specified otherwise. Elemental analyses were performed by Quantitative Technologies, Inc., Whitehouse, N.J. Water content (KF) was determined by Karl Fisher titration on a Metrohm 737 KF Coulometer.

### Example 1

### Preparation of 1-Methanesulfonyl-4-4-nitro-3-trimethylsilylmethyl-benzyl)-piperazine (3)

Method A: To a solution of 7.00 g (23.4 mmol) of 2 in 130 mL of THF at −25° C. was added dropwise 30 mL (30.4 mmol, 1M solution in THF) of trimethylsilylmethyl-magnesium chloride at such a rate that the internal temperature did not rise above −15° C. After stirring at −20° C. for 15 min, 6.4 g (28.2 mmol) of DDQ in 20 mL of THF was added and the reaction mixture allowed to warm to 10° C. over 2 h. The reaction mixture was diluted with 100 mL of isopropyl acetate and washed with 100 mL sat. NaHCO₃ (3×) and concentrated under reduced pressure to afford 7.85 g (95%) of 6 as a dark solid which could be used in the next reaction without any further purification. An analytical sample could be obtained by crystallization from EtOAc/hexane: mp 57-58° C.;

¹H NMR (CDCl₃, 400 MHz) δ-0.01 (s, 9H), 2.57 (m, 4H), 2.59 (s, 2H), 2.79 (s, 3H), 3.25 (m, 4H), 3.55 (s, 2H), 7.11 (s, 1H), 7.16 (d, 1H, J=8.4 Hz), 7.90 (d, 1H, J=8.4 Hz); ¹³C NMR (CDCl₃, 100 MHz) δ-1.40, 25.0, 34.4, 45.9, 52.4, 61.7, 125.3, 125.5, 131.7, 137.8, 143.2, 146.9; Anal. Calcd. For C₁₆H₂₇N₃O₄SSi: C, 49.84; H, 7.06; N, 10.90. Found: C, 49.62; H, 7.08; N, 10.82.

Method B: To a 5.0 L 4-neck flask equipped with a thermocouple and overhead stirrer was added 1.0 L of THF followed by 2 (184.1 g, 0.615 mol). The sides of the reaction flask were rinsed with an additional 0.20 L of THF (the starting material is not totally soluble). The reaction mixture was cooled to −20° C. (more starting material comes out of solution) and trimethylsilylmethylmagensium chloride (1.0 M in Et₂O, 0.800 L) was added dropwise at such a rate that the internal temperature did not rise above −5° C. The mixture was aged for 30 min and then poured directly into 0.800 L of 1 M aqueous I₂ solution and the resultant mixture was aged for 3 h at rt. To the reaction mixture was added 0.300 L of 0.3 M Na₂S₂O₃ pentahydrate and 1.2 L of IPAC, The aqueous layer was cut. The organic layer was washed with 0.500 L of water and then 0.500 L of brine. The IPAC layer was azeotropically dried to a Kf below 200 and a final volume of 1.3 L in IPAC for use in the next reaction. Assay amount of 3: 170 g (72%).

### Example 2

### Preparation of 2-Methoxy-quinoline-3-carboxaldehyde (4)

To a solution of 5 g (75.7 mmol) of KOH in 100 mL of MeOH was added 10 g (52.2 mmol) of 2-chloro-3-quinolinecarboxaldehyde. The mixture was heated to reflux for 2.5 h and then cooled to rt. To the solution was added 300 mL of water and the precipitated product collected by filtration to afford 7.82 g (80%) of 4 as a tan solid. An analytical sample could be prepared by recrystallization from CH₂Cl₂/hexanes: mp 92-93° C.;

¹H NMR (CDCl₃, 400 MHz) δ4.14 (s, 3H), 7.37 (dd, 1H, J=8.0 and 6.9 Hz), 7.67 (m, 1H), 7.76 (d, 1H, J=8.0 Hz), 7.80 (d, 1H, J=8.4 Hz), 8.48 (s, 1H), 10.40 (s, 1H); ¹³C NMR (CDCl₃, 100 MHz) δ 53.8, 120.0, 124.4, 125.0, 127.3, 129.7, 132.5, 139.9, 148.9, 161.1, 189.2; Anal. Calcd. For C₁₁H₉NO₂: C, 70.58; H, 4.85; N, 7.48. Found: C, 70.44; H, 4.70; N, 7.39.

### Example 3

### Preparation of 2-[5-(4-methanesulfonyl-piperazin-1-ylmethyl)-2-nitro-phenyl]-1-(2-methoxy-quinolin-3-yl)-ethanol (5)

To a mixture of 5.03 g (13.0 mmol) of 3 and 2.44 g (13.0 mmol) of 4 in 60 mL of isopropyl acetate was added dropwise 3.3 mL (3.25 mmol) of a 1 M solution of TBAF. After 30 min the reaction mixture was diluted with 35 mL of isopropyl acetate and washed with 50 mL of sat. NH₄Cl and 50 mL of water. The organic layer was dried over MgSO₄ and concentrated under reduced pressure to give 5.80 g (89%) of 5 as a colorless foam which was used in the next step without further purification. An analytical sample was obtained by chromatography on silica gel:

¹H NMR (CDCl₃, 400 MHz) δ 2.35 (m, 4H), 2.75 (s, 3H), 3.06 (m, 5H), 3.50 (m, 4H), 5.28 (t, 1H, J=6.0 Hz), 7.11 (s, 1H), 7.29 (dd, 1H, J=8.3 and 1.8 Hz), 7.38 (m, 1H), 7.60 (m, 1H), 7.83 (d, 1H, J=8.3 Hz), 7.90 (d, 1H, J=8.4 Hz), 7.96 (s, 1H); ¹³C NMR (CDCl₃, 100 MHz) δ 34.3, 40.4, 45.8, 52.2, 53.6, 61.5, 70.3, 124.5, 125.0, 125.2, 126.9, 127.2, 127.5, 127.8, 129.6, 133.2, 133.4, 135.1, 143.3, 145.8, 149.2, 159.4; Anal. Calcd. For C₂₄H₂₈N₄O₆S.½H₂O: C, 56.57; H, 5.74; N, 10.99. Found: C, 56.65; H, 5.44; N, 10.83.

### Example 4

### Trans-3-{2-[5-(4-methanesulfonyl-piperazine-1-ylmethyl)-2-nitro-phenyl]-vinyl}-2-methoxy-quinoline (1)

To a solution of 3.50 g (7.00 mmol) of 5 in 50 mL of THF was added 4.41 g (21.0 mmol) of trifluoroacetic acid. After stirring for 30 min at rt, DBU (6.39 g, 42.0 mmol) was added and the mixture heated 50° C. for 1 h. The reaction mixture was concentrated under reduced pressure and the residue purified by silica gel chromatography to give 2.64 g (78%) of 1 as a yellow solid: mp 156-157° C.;

¹H NMR (CDCl₃, 400 MHz) δ 2.63 (m, 4H), 2.81 (s, 3H), 3.31 (m, 4H), 3.67 (s, 2H), 4.18 (s, 3H), 7.42 (m, 3H), 7.63 (dt, 1H, J=6.9 and 1.4 Hz), 7.83 (m, 4H), 7.98 (d, 1H, J=8.4 Hz), 8.24 (s, 1H); ¹³C NMR (CDCl₃, 100 MHz) δ 34.5, 45.9, 52.5, 53.8, 61.8, 121.8, 124.6, 125.2, 125.4, 126.4, 127.0, 127.8, 127.9, 128.5, 128.6, 129.9, 133.4, 135.1, 143.9, 146.3, 147.1, 159.8; Anal. Calcd. For C₂₄H₂₆N₄O₅S: C, 59.74; H, 5.43; H, 11.61. Found: C, 59.51; H, 5.17; N, 11.53.

The following is the X-ray powder diffraction pattern having diffraction angles (°) of: 9.5, 15.7, 16.8, 17.3, 18.4, 18.8, 21.0, 22.5, and 23.7.

### Example 5

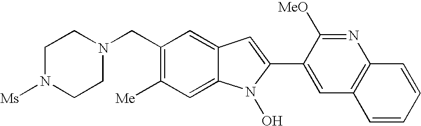 2-(2-methoxyquinolin-3-yl)-6-methyl-5-{[4-(methylsulfonyl)piperazin-1-yl]methyl})-1H-indol-1-ol (Compound B)

A glass tube was charged with 1 (100 mg, 0.207 mmol), toluene (3 mL), phen₂Pd(OAc)₂ (1.47×10⁻³M solution in toluene, 0.141 mL, 2.07×10⁻⁴ mmol). The tube was placed in an Endeavor reactor and purged three times successively with N₂ and CO. The vessel was pressurized to 15 psig with CO and aged at 70° C. for 16 h. After cooling to rt, the reaction mixture was concentrated in vacuo. Purification by silical gel chromatography afforded Compound B as an off-white solid (0.020 g, 20% yield).

¹H NMR (CDCl₃, 400 MHz) δ 1.85 (m, 4H), 2.52 (m, 5H), 2.60 (s, 3H), 3.07 (m, 4H), 3.58 (s, 2H), 3.74 (m, 4H), 4.21 (s, 3H), 6.65 (d, 1H, J=0.5 Hz), 7.17 (dd, 1H, J=8.1 and 1.4 Hz), 7.44 (m, 3H), 7.67 (ddd, 1H, J=8.4, 7.0 and 1.4 Hz), 7.78 (dd, 1H, J=8.1 and 0.9 Hz), 7.91 (d, 1H, J=8.4 Hz), 8.39 (s, 1H); ¹³C NMR (CDCl₃, 100 MHz) δ 25.7, 33.9, 45.7, 52.1, 54.4, 63.1, 68.0, 99.7, 108.9, 115.9, 121.7, 123.4, 124.5, 125.0, 125.3, 127.2, 127.7, 128.2, 130.2, 133.4, 134.5, 139.3, 145.9, 158.5. Anal. Calcd. For C₂₅H₂₈N₄O₄S.THF: C, 62.43; H, 6.36; N, 10.40. Found: C, 62.27; H, 6.40; N, 10.04.

### Example 6

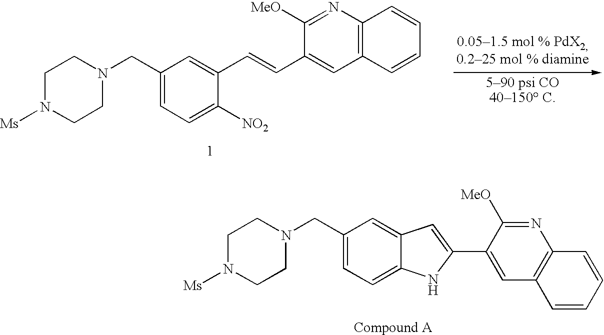

### 2-Methoxy-3-[5-[[4-methysulfonyl)-1-piperazinyl]methyl]-1H-indol-2-yl]-quinoline (Compound A)

Method A: An autoclave was charged with 1 (15 g, 31.1 mmol), palladium (II) trifluroacetate (0.020 g, 0.062 mmol), 3,4,7,8-tetramethyl-1,10-phenathroline (0.102 g, 0.435 mmol), and DMF (100 mL). The vessel was purged three times successively with N₂ and CO. The reactor was pressurized to 15 psig with CO and aged at 70° C. for 14 h. The reaction mixture was filtered through solka floka. The filtrate was concentrated to 40 mL and heated to 50° C. MeOH (20 mL) was added and the mixture was allowed to cool to rt. The product was isolated as a pale yellow solid (11.63 g, 83% yield): mp 197-198° C.;

¹H NMR (CDCl₃, 400 MHz) δ 2.61 (m, 4H), 2.78 (s, 3H), 3.27 (m, 4H), 3.66 (s, 2H), 4.31 (s, 3H), 7.07 (s, 1H), 7.18 (dd, 1H, J=8.3 and 1.4 Hz), 7.44 (m, 2H), 7.57 (s, 1H), 7.64 (t, 1H, J=8.4 Hz), 7.81 (d, 1H, J=8.1 Hz), 7.88 (d, 1H, J=8.4 Hz), 8.48 (s, 1H), 9.68 (br s, 1H); ¹³C NMR (CDCl₃, 100 MHz) δ 34.0, 46.0, 52.3, 54.1, 63.3, 101.5, 111.3, 116.8, 121.1, 124.2, 124.8, 125.5, 127.0, 127.6, 128.3, 129.0, 129.6, 134.0, 135.2, 136.0, 145.3, 158.3; Anal. Calcd. For C₂₄H₂₆N₄O₃S: C, 63.98; H, 5.82; N, 12.44. Found: C, 64.28; H, 5.68; N, 12.05.

The following is the X-ray powder diffraction pattern having diffraction angles (°) of: 7.8, 9.1, 13.0, 14.7, 14.9, 16.1, 16.7, 18.1, 19.6, 20.9, 21.1, and 22.4.

Method B: A glass tube was charged with 1 (100 mg, 0.207 mmol), DMF (3 mL), Pd(II) trifluoroacetate (9.63×10⁻⁴M solution in DMF, 0.215 mL, 2.07×10⁻⁴ mmol) and 1,10-phenanthroline (1.66×10⁻²M solution in DMF, 0.312 mL, 5.18×10⁻³ mmol). The tube was placed in an Endeavor reactor and purged three times successively with N₂ and CO. The vessel was pressurized to 15 psig with CO and aged at 70° C. for 16 h. After cooling to rt, HPLC analysis indicated the formation of Compound A in 95% yield.

Method C: A glass tube was charged with Compound B (97 mg, 0.207 mmol), DMF (2.4 mL) and phen₂Pd(OAc)₂ (3.56×10⁻³M solution in DMF, 1.0 mL, 3.56×10⁻³ mmol). The tube was placed in an Endeavor reactor and purged three times successively with N₂ and CO. The vessel was pressurized to 15 psig with CO and aged at 70° C. for 16 h. After cooling to rt, HPLC analysis indicated the formation of Compound A in 79% yield.

Additional compounds may be synthesized utilizing the procedures described above, by substituting the appropriate styrene, as depicted below in Table 1.

TABLE 1
Reductive Cyclization of ortho-Nitrostyrenes

| styrene | product |
|---|---|
| 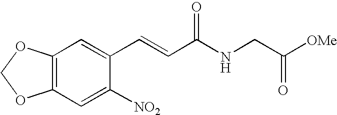 | 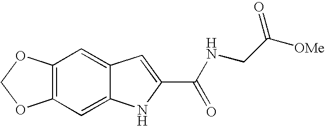 |
| Methyl-N-[(2E)-3-(6-nitro-1,3-benzodioxol- | N-(Carbomethoxy)-5,6-methylenedioxy-1H- |
| 5-yl)prop-2-enoyl]glycinate | indole-2-carboxamide |
| 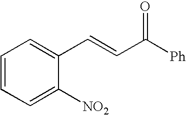 | 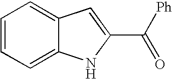 |
| (2E)-3-(2-nitrophenyl)-1-phenylprop-2-en-1- | 2-Benzoyl-1H-indole |
| one | |
| 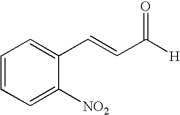 | 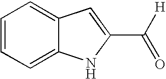 |
| (2E)-3-(2-nitrophenyl)acrylaldehyde | Indole-2-carboxaldehyde |
| 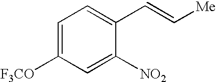 | 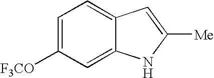 |
| 2-Nitro-1-[(1E)-prop-1-en-1-yl]-4- | 2-Methyl-7-trifluoromethyl indole |
| (trifluoromethoxy)benzene | |
| 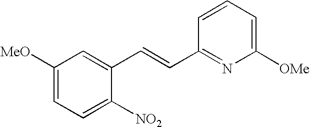 | 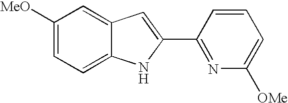 |
| 2-Methoxy-5-[(E)-2-(5-methoxy-2- | 2-Methoxy-6-[5-methoxy-1H-indol-2-yl] |
| nitrophenyl)vinyl]pyridine | pyridine |
| 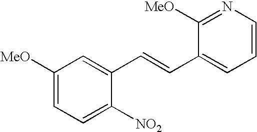 | 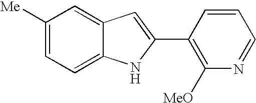 |
| 2-Methoxy-3-[(E)-2-(5-methyl-2- | 2-Methoxy-3-[5-methyl-1H-indol-2yl] pyridine |
| nitrophenyl)vinyl]pyridine | |
| 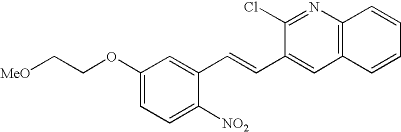 | 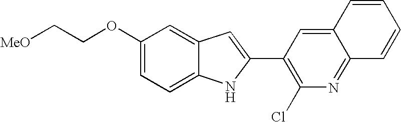 |
| 2-Chloro-3-[(E)-)-2-[5-(2-methoxyethoxy)-2- | 2-Chloro-3-[5-(methoxyethoxy)-1H-indol-2- |
| nitrophenyl]vinyl}quinoline | yl]quinoline |
| 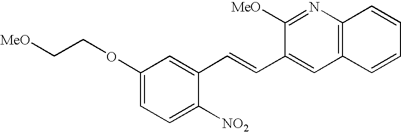 | 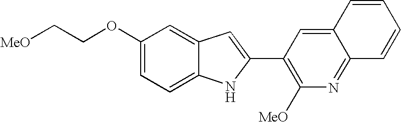 |
| 2-Methoxy-3-{(E)-2-[5-(2-methoxyethoxy)- | 2-Methoxy-3-[5-(methoxyethoxy)-1H-indol-2- |
| 2-nitrophenyl]vinyl} quinoline | yl]quinoline |
| 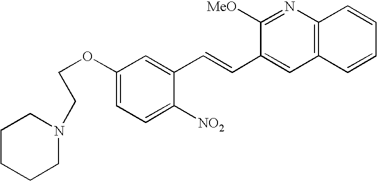 | 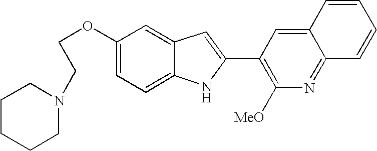 |
| 2-Methoxy-3-{(E)-2-[5-(2-methoxyethoxy)- | 2-Methoxy-3-[5-(1-piperdinylethoxy)-1H-indol- |
| 2-nitrophenyl]vinyl} quinoline | 2-yl]quinoline |
| 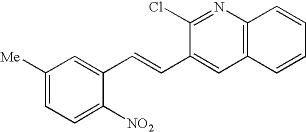 | 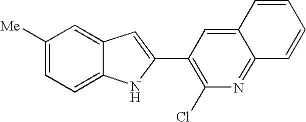 |
| 2-Chloro-3-[(E)-2-(5-methyl-2- | 2-Chloro-3-(5-methyl-1H-indol-2-yl)quinoline |
| nitrophenyl)vinyl]quinoline | |
| 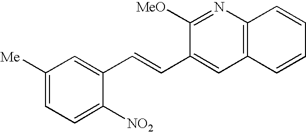 | 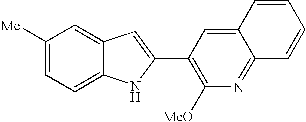 |
| 2-Methoxy-3-[(E)-2-(5-methyl-2- | 2-Methoxy-3-(5-methyl-1H-indol-2- |
| nitrophenyl)vinyl]quinoline | yl)quinoline |
| 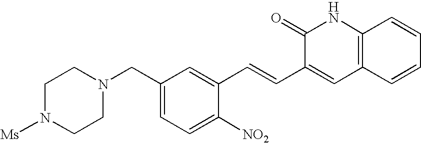 | 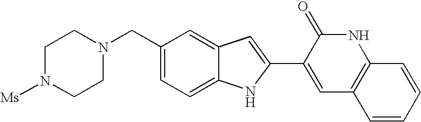 |
| 3-[(E)-2-(5-{[4-(methylsulfonyl) piperazin- | 3-[5-[4-(Methylsulfonyl)-1- |
| 1-yl]methyl}-2-nitrophenyl)vinyl]quinolin-2- | piperazinyl]methyl]-1H-indole-2-yl]quinolin- |
| (1H)-one | 2(1H)-one |
| 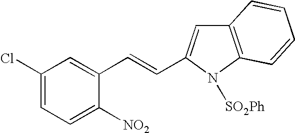 | 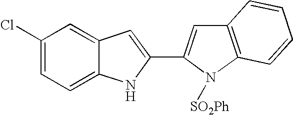 |
| | 1-Benzenesulfonyl-2-(1′benzyl-5-chloroindol- |
| | 2′-yl) indole |
| 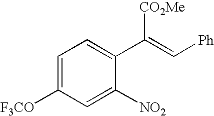 | 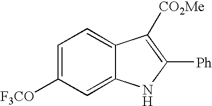 |
| 2-[(E)-2-(5-chloro-2-nitrophenyl)vinyl]-1- | Methyl 2-phenylindole-3-carboxylate |
| (phenylsulfonyl)-1H-indole | |
| 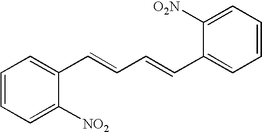 | 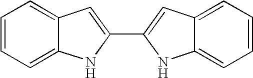 |
| 1,1′-(1E,3E)-buta-1,3-diene-1,4-diylbis (2- | 2,2′-Bisindole |
| nitrobenzene) | |

### Example 7

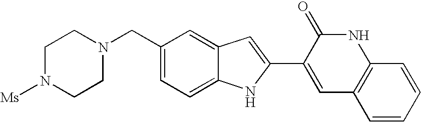

### 3-(5{[4-(Methylsulfonyl)piperzin-1-yl]methyl}-1H-indol-2-yl)quinolin-2(1H-one

To an 80° C. solution of HCl (9 N, 1.7 mL, 15 mmol) in DMAc (2 mL) was charged a solution of Compound A (450 mg, 1 mmol) in DMAc (3 mL). After 2 h, the mixture was cooled to 60° C., and EtOH (15 mL) was added. The mixture was allowed to cool to rt. The mixture was filtered to afford the title product as a yellow solid (390 mg, 90%). m.p. 275-277° C.;

¹H NMR (DMSO-d⁶, 400 MHz) δ 12.12 (1H, s), 11.50 (1H, s), 8.49 (1H, 1), 7.69 (1H, d, J=0.6 Hz), 7.47 (1H, d, J=8.3 Hz), 7.46 (1H, dd, J=5.4 Hz, XXHz), 7.43 (1H,s), 7.35 (1H, d, J=8.2 Hz), 7.25 (1H, d, J=1.1 Hz), 7.21 (1H, t J=XX Hz), 7.05 (1H, d, J=9.1 Hz), 3.53 (2H, s), 3.07 (4H, s), 2.82 (3H, s), 2.47 (4H, s); ¹³C NMR (DMSO-d⁶, 100 MHz) δ, Anal. Calcd for C₂₃H₂₄N₄O₃S: C, 63.28; H, 5.54; N, 12.83; O, 11.0; S, 7.35. Found: C, 62.99; H, 5.56; N, 12.68; 0, 11.28, S, 7.02.

What is claimed is:

1. A compound of Formula I: 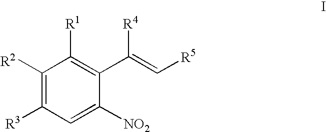
wherein
R^{a} is independently selected from a) hydrogen, and b) unsubstituted or substituted C₁-C₆ alkyl;
R¹ is a) hydrogen, b) unsubstituted or substituted C₁-C₆ alkyl, and c) OR⁷;
R² is a) hydrogen, b) unsubstituted or substituted C₁-C₆ alkyl, c) CR^{a}₂)ₙR⁷, d) O(CR^{a}₂)ₙOR⁷, e) O(CR^{a}₂)ₙR⁷, or f) halo;
R³ is a) hydrogen, b) unsubstituted or substituted C₁-C₆ alkyl, or c) OR⁷;
R² and R³ can be taken together to form a cyclic moiety, (CH₂)ᵤ, said cyclic moiety optionally containing one or two heteroatoms selected from N, O and S;
R⁴ is a) hydrogen, b) unsubstituted or substituted C₁-C₆ alkyl, c) OR⁷, or d) C(O)₂R⁷;
R⁵ is a) unsubstituted or substituted C₁-C₆ alkyl, b) C₂-C₆ alkenyl-R⁷, c) C₂-C₆ alkynyl-R⁷, d) unsubstituted or substituted aryl, e) unsubstituted or substituted heterocyclyl, f) C(O)NR⁷CR^{a}₂)ₙC(O)OR⁷, or g) C(O)R⁷; said alkyl, alkenyl, alkynyl, aryl or heterocyclyl is optionally substituted with at least one substituent selected from: i) halo, ii) unsubstituted or substituted C₁-C₆ alkyl, iii) OR⁷, iv) NR⁷₂, v) NO₂, and vi) S(O)ₘR⁶;
R⁶ is independently selected from a) unsubstituted or substituted C₁-C₆ alkyl, and b) unsubstituted or substituted aryl;
R⁷ is independently selected from a) H, b) unsubstituted or substituted C₁-C₆ alkyl, c) unsubstituted or substituted aryl, d) unsubstituted or substituted heterocyclyl, and e) CF₃; said alkyl, aryl and heterocyclyl is optionally substituted with at least one substituent selected from i) halo, ii) unsubstituted or substituted C₁-C₆ alkyl, iii) OR⁷, iv) NR⁷₂, v) NO₂, and vi) S(O)ₘR⁶,
m is 1 or 2;
n is independently 0, 1, 2, 3, or 4;
u is 4, 5, 6, 7 or 8;
or a salt thereof.

2. The compound according to claim 1, wherein:
R¹ is hydrogen;
R⁴ is a) hydrogen, or b) C(O)₂R⁷;
or a salt thereof.

3. The compound of claim 1 selected from:
Trans-3-{2-[5-(4-methanesulfonyl-piperazine-1-ylmethyl)-2-nitro-phenyl]-vinyl}-2-methoxy-quinoline;
Methyl-N-[(2E)-3-(6-nitro-1,3-benzodioxol-5-yl)prop-2-enoyl]glycinate;
(2E)-3-2-nitrophenyl)-1-phenylprop-2-en-1-one;
(2E)-3-(2-nitrophenyl)acrylaldehyde;
2-Nitro-1-[(1E)-prop-1-en-1-yl]4-(trifluoromethoxy)benzene;
2-Methoxy-5-[(E)-2-(5-methoxy-2-nitrophenyl)vinyl]pyridine;
2-Methoxy-3-[(E)-2-(5-methyl-2-nitrophenyl)vinyl]pyridine;
2-Chloro-3-[(E)-)-2-[5-2-methoxyethoxy)-2-nitrophenyl]vinyl}quinoline;
2-Methoxy-3-{(E)-2-[5-(2-methoxyethoxy)-2-nitrophenyl]vinyl}quinoline;
2-Methoxy-3-[(E)-2-[2-nitro-5-(2-piperidin-1-ylethoxy)phenyl]vinyl}quinoline;
2-Chloro-3-[(E)-2-(5-methyl-2-nitrophenyl)vinyl]quinoline;
2-Methoxy-3-[(E)-2-5-methyl-2-nitrophenyl)vinyl]quinoline;
3-[(E)-2-(5-{[4-(methylsulfonyl)piperazin-1-yl]methyl}-2-nitrophenyl)vinyl]quinolin-2-(1H)-one;
2-[(E)-2-(5-chloro-2-nitrophenyl)vinyl]-1-(phenylsulfonyl)-1H-indole;
Methyl (2Z)-2-[2-nitro-4-(trifluoromethoxy)phenyl]-3-phenylacrylate;
1,1′-1E,3E)-buta-1,3-diene-1,4-diylbis(2-nitrobenzene);
or a salt thereof.

4. A compound of Formula II: 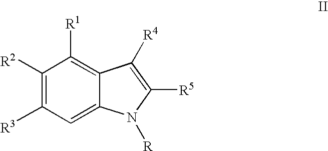
wherein
R is H or OH;
R^{a} is independently selected from a) hydrogen, and b) unsubstituted or substituted C₁-C₆ alkyl;
R¹ is a) hydrogen, b) unsubstituted or substituted C₁-C₆ alkyl, and c) OR⁷;
R² is a) hydrogen, b) unsubstituted or substituted C₁-C₆ alkyl, c) CR^{a}₂)ₙR⁷, d) O(CR^{a}₂)ₙOR⁷, e) O(CR^{a}₂)ₙR⁷, or f) halo;
R³ is a) hydrogen, b) unsubstituted or substituted C₁-C₆ alkyl, or c) OR⁷;
R² and R³ can be taken together to form a cyclic moiety, (CH₂)ᵤ, said cyclic moiety optionally containing one or two heteroatoms selected from N, O and S;
R⁴ is a) hydrogen, b) unsubstituted or substituted C₁-C₆ alkyl, c) OR⁷, or d) C(O)₂R⁷;
R⁵ is a) unsubstituted or substituted C₁-C₆ alkyl, b) C₂-C₆ alkenyl-R⁷, c) C₂-C₆ alkynyl-R⁷, d) unsubstituted or substituted aryl, e) unsubstituted or substituted heterocyclyl, or f) C(O)NR⁷CR^{a}₂)ₙC(O)OR⁷; said alkyl, alkenyl, alkynyl, aryl or heterocyclyl is optionally substituted with at least one substituent selected from: i) halo, ii) unsubstituted or substituted C₁-C₆ alkyl, iii) OR⁷, iv) NR⁷₂, v) NO₂, and vi) S(O)ₘR⁶;
R⁶ is independently selected from a) unsubstituted or substituted C₁-C₆ alkyl, and b) unsubstituted or substituted aryl;
R⁷ is independently selected from a) H, b) unsubstituted or substituted C₁-C₆ alkyl, c) unsubstituted or substituted aryl, d) unsubstituted or substituted heterocyclyl, and e) CF₃; said alkyl, aryl and heterocyclyl is optionally substituted with at least one substituent selected from i) Halo, ii) unsubstituted or substituted C₁-C₆ alkyl, iii) OR⁷, iv) NR⁷₂, v) NO₂, and vi) S(O)ₘR⁶,
m is 1 or 2;
n is independently 0, 1, 2, 3, or 4;
u is 4, 5, 6, 7 or 8;
or a pharmaceutically acceptable salt thereof.

5. The compound according to claim 4 wherein:
R¹ is hydrogen;
R⁴ is hydrogen or C(O)₂R⁷;
R⁵ is a) unsubstituted or substituted C₁-C₆ alkyl, b) unsubstituted or substituted aryl, c) unsubstituted or substituted heterocyclyl, or d) C(O)NR⁷CR^{a}₂)ₙC(O)OR⁷;
or a pharmaceutically acceptable salt thereof.

6. The compound according to claim 5 selected from:
2-Methoxy-3-[5-(piperazin-1-ylmethyl)-1H-indol-2-yl]quinoline;
N-(Carbomethoxy)-5,6-methylenedioxy-1H-indole-2-carboxamide;
2-(2-methoxyquinolin-3-yl)-6-methyl-5-{[4-methylsulfonyl)piperazin-1-yl]methyl}-1H-indol-1-ol;
2-Methoxy-6-[5-methoxy-1H-indol-2-yl] pyridine;
2-Methoxy-3-[5-methyl-1H-indol-2-yl] pyridine;
2-Chloro-3-[5-(methoxyethoxy)-1H-indol-2-yl]quinoline;
2-Methoxy-3-[5-(methoxyethoxy)-1H-indol-2-yl]quinoline;
2-Methoxy-3-[5-(1-piperdinylethoxy)-1H-indol-2-yl]quinoline;
2-Chloro-3-5-methyl-1H-indol-2-yl)quinoline;
2-Methoxy-3-(5-methyl-1H-indol-2-yl)quinoline;
3-[5-[4-(Methylsulfonyl)-1-piperazinyl]methyl)-1H-indole-2-yl]quinolin-2(1H)-one;
1-Benzenesulfonyl-2-(1′benzyl-5-chloroindol-2′-yl) indole;
Methyl 2-phenylindole-3-carboxylate;
or a pharmaceutically acceptable salt thereof.

7. A compound selected from:
2-(2-methoxyquinolin-3-yl)6-methyl-5-{[4-(methylsulfonyl)piperazin-1-yl]methyl}-1H-indol-1-ol; and
2-Methoxy-3-[5-[[4-(methysulfonyl)-1-piperazinyl]methyl]-1H-indol-2-yl]-quinoline
or a pharmaceutically acceptable salt thereof.

8. A process for preparing the compound of the Formula II, according to claim 4, which comprises a palladium-catalyzed reductive cyclization of an ortho-nitrostyrene of Formula I: 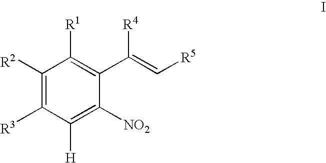
wherein
R^{a} is independently selected from a) hydrogen, and b) unsubstituted or substituted C₁-C₆ alkyl;
R¹ is a) hydrogen, b) unsubstituted or substituted C₁-C₆ alkyl, and c) OR⁷;
R² is a) hydrogen, b) unsubstituted or substituted C₁-C₆ alkyl, c) CR^{a}₂)ₙR⁷, d) O(CR^{a}₂)ₙOR⁷, e) O(CR^{a}₂)ₙR⁷, or f) halo;
R³ is a) hydrogen, b) unsubstituted or substituted C₁-C₆ alkyl, or c) OR⁷;
R² and R³ can be taken together to form a cyclic moiety, (CH₂)ᵤ, said cyclic moiety optionally containing one or two heteroatoms selected from N, O and S;
R⁴ is a) hydrogen, b) unsubstituted or substituted C₁-C₆ alkyl, c) OR⁷, or d) C(O)₂R⁷;
R⁵ is a) unsubstituted or substituted C₁-C₆ alkyl, b) C₂-C₆ alkenyl-R⁷, c) C₂-C₆ alkynyl-R⁷, d) unsubstituted or substituted aryl, e) unsubstituted or substituted heterocyclyl, f) C(O)NR⁷(CR^{a}₂)ₙC(O)OR⁷ or g) C(O)R⁷; said alkyl, alkenyl, alkynyl, aryl or heterocyclyl is optionally substituted with at least one substituent selected from: i) halo, ii) unsubstituted or substituted C₁-C₆ alkyl, iii) OR⁷, iv) NR⁷₂, v) NO₂, and vi) S(O)ₘR⁶;
R⁶ is independently selected from a) unsubstituted or substituted C₁-C₆ alkyl, and b) unsubstituted or substituted aryl;
R⁷ is independently selected from a) H, b) unsubstituted or substituted C₁-C₆ alkyl, c) unsubstituted or substituted aryl, d) unsubstituted or substituted heterocyclyl, and e) CF₃; said alkyl, aryl and heterocyclyl is optionally substituted with at least one substituent selected from i) halo, ii) unsubstituted or substituted C₁-C₆ alkyl, iii) OR⁷, iv) NR⁷₂, v) NO₂, and vi) S(O)ₘR⁶,
m is 1 or 2;
n is independently 0, 1, 2, 3, or 4;
u is 4, 5, 6, 7 or 8;
to produce a compound of Formula II.

9. The process of claim 8, wherein the palladium catalyst is generated in situ.

10. The process of claim 9 wherein the palladium catalyst is comprised of a palladium source, which is selected from palladium (II) acetate, palladium (II) trifluoroacetate and Pd₂(dba)₃, and a ligand, which is selected from an aromatic diamine.

11. The process of claim 10, wherein the aromatic diamine is selected from 1,10-phenanthroline (phen), 3,4,7,8-tetramethyl-1,10-phenanthroline and bipyridine.

12. The process of claim 11 wherein the palladium is about 0.05 to about 1.5 mol % and the ligand is about 0.2 to about 25 mol %.

13. The process of claim 8 wherein the palladium catalyst is preformed and is selected from phen₂Pd(OTf)₂, phen₂Pd(PF₆)₂ and phen₂Pd(BF₄)₂.

14. The process of claim 13 which further comprises an additive, which is selected from Ag(OTf)₂ and Cu(OAc)₂.

15. The process of claim 14 which further comprises a solvent selected from dimethylformamide, DMSO, THF, acetonitrile, toluene, dimethylacetamide, N-methyl pyrrolidinone, and ortho-dichlorobenzene.

16. The process of claim 11 wherein the palladium catalyst is palladium (II) trifluoracetate, the aromatic diamine is 3,4,7,8-tetramethyl-1,10-phenathroline, and a solvent is added.

17. The process of claim 16 wherein the pressure is about 15 psig CO and the temperature is about 70° C.

18. A process for preparing 2-(2-methoxyquinolin-3-yl)-6-methyl-5-{[4-(methylsulfonyl)piperazin-1-yl]methyl}-1H-indol-1-ol which comprises
a) mixing trans-3-{2-[5-(4-methanesulfonyl-piperazine-1-ylmethyl)-2-nitro-phenyl]-vinyl}-2-methoxy-quinoline with a palladium catalyst and a solvent to produce a reaction mixture;
b) pressurizing the reaction mixture to about 15 psig with CO and maintaining a temperature of about 70° C.; and
c) isolating 2-(2-methoxyquinolin-3-yl)-6-methyl-5-{[4-(methylsulfonyl)piperazin-1-yl]methyl}-1H-indol-1-ol.

19. A process for preparing 2-methoxy-3-[5-[[4-(methysulfonyl)-1-piperazinyl]methyl]-1H-indol-2-yl]-quinoline which comprises
a) mixing trans-3-{2-[5-(4-methanesulfonyl-piperazine-1-ylmethyl)-2-nitro-phenyl]-vinyl}-2-methoxy-quinoline with a palladium catalyst, a aromatic diamine and a solvent to produce a reaction mixture;
b) pressurizing the reaction mixture to about 15 psig with CO and maintaining a temperature of about 70° C.; and
c) isolating 2-methoxy-3-[5-[[4-methysulfonyl)-1-piperazinyl]methyl]-1H-indol-2-yl]-quinoline.
